(19) United States
(12) Patent Application Publication
(10) Pub. No.: US 2019/0038248 A1
(43) Pub. Date: Feb. 7, 2019

(54) Veterinary X-ray Sensor and Boot

(71) Applicant: Dentalaire Products International, Irvine, CA (US)

(72) Inventor: Timothy P. Martelon, Barrington, IL (US)

(73) Assignee: Dentalaire Products Int'l, Irvine, CA (US)

(21) Appl. No.: 16/053,033

(22) Filed: Aug. 2, 2018

Related U.S. Application Data
(60) Provisional application No. 62/541,369, filed on Aug. 4, 2017.

(51) Int. Cl. A61B 6/00; A61B 6/14

(52) U.S. Cl. CPC A61B 6/508; A61B 6/44; A61B 6/14

(57) ABSTRACT

Elastomeric positioning aides, veterinary dental sensors, and veterinary dental processes are improved by the use of a silicon rubber boot. The silicon rubber boot includes an interior surface, an exterior surface and an orifice connecting the exterior surface to the interior surface. The orifice is adapted to elastically expand and pass a digital x-ray sensor therethrough whereafter the digital x-ray sensor is adjacent to the interior surface and has a connection to a digital recording system passing through the orifice. The interior surface is adapted to adhere to an exposure surface of the digital x-ray sensor and to grip edges of the digital x-ray sensor, and the exterior surface adapted to grip a tooth of a veterinary patient and provide visual identification of the digital x-ray sensor within the veterinary patient's mouth. The veterinary dental sensor includes a digital x-ray sensor and the veterinary dental process includes the use of the veterinary dental sensor.

### CROSS-REFERENCE TO RELATED APPLICATIONS

This disclosure claims the benefit of priority to U.S. Provisional Patent application Ser. No. 62/541,369, filed Aug. 4, 2017, which is incorporated herein in its entirety.

### FIELD OF THE INVENTION

This invention relates to protective elements for digital x-ray sensors used in veterinary dentistry.

### BACKGROUND

Dental sensors used for veterinary medicine can be damaged if the patient awakes from anesthesia or if there is nerve stimulation. A bisecting angle technique is used in veterinary dentistry which makes the dental sensor more difficult to position in the patient's mouth.

Currently, there is a product (a plastic bag) that is slipped over the entire sensor and cable. The device that is slipped over the sensor is bulky, does not fit tight, does not provide adequate protection and is more difficult to position in the mouth.

As can be seen, there is a need for improved housing for digital x-ray sensors in veterinary dentistry.

### SUMMARY

A first embodiment is an elastomeric positioning aide that includes a silicon rubber boot that includes an interior surface, an exterior surface and an orifice connecting the exterior surface to the interior surface, the orifice adapted to elastically expand and pass a digital x-ray sensor therethrough whereafter the digital x-ray sensor is adjacent to the interior surface and has a connection to a digital recording system passing through the orifice, the interior surface adapted to adhere to an exposure surface of the digital x-ray sensor and to grip edges of the digital x-ray sensor, the exterior surface adapted to grip a tooth of a veterinary patient and provide visual identification of the digital x-ray sensor within the veterinary patient's mouth.

A second embodiment is a veterinary dental sensor that includes a digital x-ray sensor having an exposure surface, a plurality of perimeter surfaces, and a connection surface opposite the exposure surface and adapted to communication with a digital recordation system; an elastomeric positioning aide covering the exposure surface and the plurality of perimeter surfaces; the elastomeric positioning aide frictionally adhered to the sensor; the elastomeric positioning aide having an internal surface adapted to receive the digital x-ray sensor and an external surface adapted to grip a tooth of a veterinary patient.

A third embodiment is a veterinary dental that includes positioning a veterinary dental sensor comprising elastomeric positioning aide covering a portion of a digital x-ray sensor against a tooth of a veterinary patient, where the elastomeric positioning aide holds the position of the veterinary dental sensor; exposing the veterinary dental sensor to x-rays to image the tooth; and then withdrawing the veterinary dental sensor from the veterinary patient.

### BRIEF DESCRIPTION OF THE FIGURES

For a more complete understanding of the disclosure, reference should be made to the following detailed description and accompanying drawing figures wherein:

FIG. 1 is a perspective image of a protective housing (elastomeric positioning aide) about a veterinary digital x-ray sensor (not showing the connection to the digital recording device);

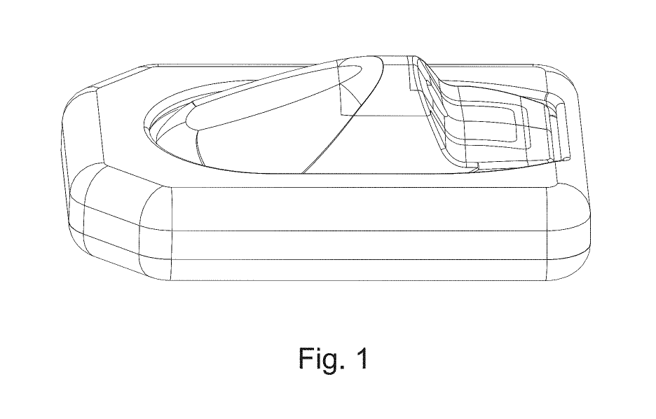

FIG. 2 is a top down view of the protective housing showing in FIG. 1;

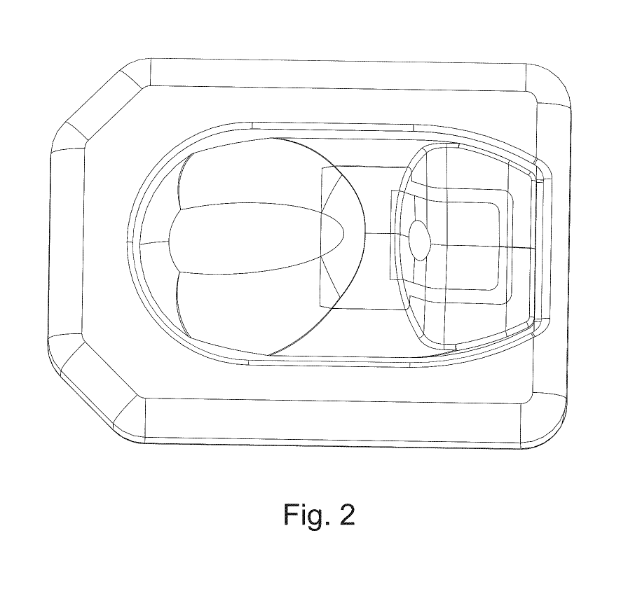

FIG. 3 is a photograph of the protective housing (elastomeric positioning aide) next to a veterinary digital x-ray sensor; and

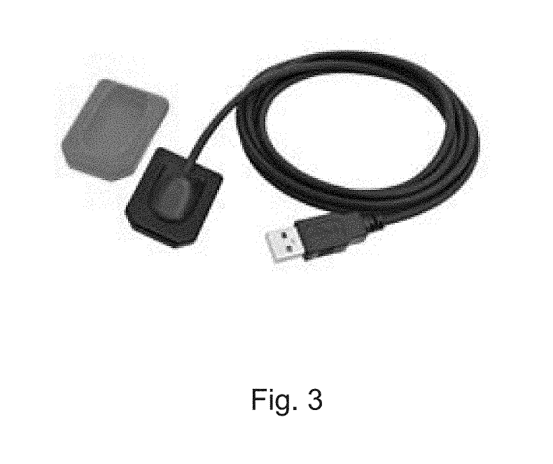

FIG. 4 is a photograph of the protective housing (elastomeric positioning aide) about the veterinary digital x-ray sensor.

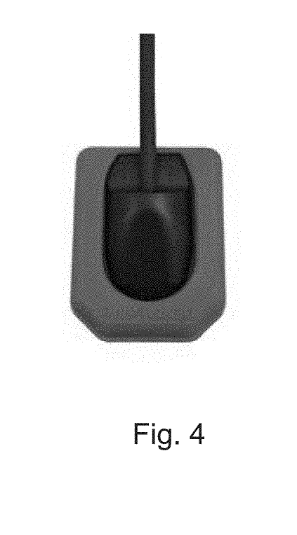

is a perspective image of a veterinary dental sensor that includes the digital x-ray sensor carrying or covered by the protective housing (elastomeric positioning aide).

While specific embodiments are illustrated in the figures, with the understanding that the disclosure is intended to be illustrative, these embodiments are not intended to limit the invention described and illustrated herein.

### DETAILED DESCRIPTION

Objects, features, and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

Herein, the use of the word “a” or “an” when used in conjunction with the term “comprising” in the claims and/or the specification may mean “one,” but it is also consistent with the meaning of “one or more,” “at least one,” and “one or more than one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

Herein is described a protective housing for digital x-ray sensors used in veterinary dentistry. These housing, alternatively described as boots or positioning aides, cover a portion of the digital x-ray sensors and provide important benefits during use, including identification in the veterinary patient's mouth, adhesion to the veterinary patient's tooth/teeth, and protection to the digital x-ray sensor from damage in the event of biting.

A first embodiment is an elastomeric positioning aide that includes a silicon rubber boot having an interior surface, an exterior surface and an orifice connecting the exterior surface to the interior surface. The orifice is adapted to elastically expand and pass a digital x-ray sensor therethrough. Whereafter the digital x-ray sensor is adjacent to the interior surface and has a connection to a digital recording system passing through the orifice. Notably, a digital x-ray sensor includes a sensor portion that has an X-ray exposure surface and a connection (wire, wires, or wire harness) to a digital recording system. The connection to the digital recording system is typically on an opposite side of the sensor from the exposure surface. Another connection can be envisioned wherein the connection extends from an edge of the sensor.

In one instance, the interior surface of the boot is adapted to adhere to an exposure surface of the digital x-ray sensor and to grip edges of the digital x-ray sensor. Preferably, the interior surface of the boot is fully adjacent to the exposure surface, that is, the interior surface contacts the entire exposure surface without a bubble or gap between the interior surface and the exposure surface. Additionally, the interior surface is preferably fully adjacent to the edges of the digital x-ray sensor, that is, the interior surfaces contact the ends of the digital x-ray sensor without a gap between them thereby preventing the slipping or movement of the boot relative to the sensor. In one example, the interior surface has interior volume dimensions that are the same as or less than 5 mm, 4 mm, 3 mm, 2 mm, or 1 mm smaller than exterior surface dimensions of the digital x-ray sensor to which it is adapted to adhere to.

In another instance, the exterior surface of the boot is adapted to grip a tooth of a veterinary patient and provide visual identification of the digital x-ray sensor within the veterinary patient's mouth. In one example, the exterior surface has an adhesive texture that aides in the positioning and retention of the position within the veterinary patient's mouth. In another example, the boot and thereby the exterior surface has a color that is atypical in a veterinary patient's mouth (for example, a bright blue color) and provides easy identification of the position of the sensor from a distance (i.e., provides the easy identification of the location of the combination of the sensor and the boot by an x-ray technician that is distal from the veterinary patient as is typical during the acquisition of the x-ray image).

Preferably, the silicon rubber boot is composed of a silicon rubber which has a Shore A Hardness of 60 to 90, more preferably about 70, a tensile strength of about 6 to about 10 N/m², more preferably about 8 to about 9 N/m², and a tear-strength of about 15 to about 30 N/m², more preferably about 20 to 25 N/m².

A second embodiment is a veterinary dental sensor that includes the digital x-ray sensor carrying or covered by an elastomeric positioning aide (i.e. boot). Typically, the digital x-ray sensor has an exposure surface, a plurality of perimeter surfaces, and a connection surface opposite the exposure surface. The digital x-ray sensor is further adapted to communicate with a digital recordation system, for example by the protrusion of a connection cable from the connection surface having, distal from the connection surface, a digital connector (e.g., a USB connection).

Preferably, the elastomeric positioning aide covers the exposure surface and the plurality of perimeter surfaces and is, preferably, frictionally adhered to the sensor (e.g. by elastomeric contraction). More preferably, the elastomeric positioning aide has an internal surface adapted to receive the digital x-ray sensor and an external surface adapted to grip a tooth of a veterinary patient.

In one instance, the elastomeric positioning aide is pigmented to a nonbiological color, for example, blue. In another instance, the elastomeric positioning aide comprises a silicon rubber. In still another instance, the elastomeric positioning aide has a Shore A Hardness of 60 to 90; of 60 to 80, of 65 to 75, or to about 70. In yet another instance, the elastomeric positioning aide has a tensile strength of about 6 to about 10 N/m², about 7 to about 9 N/m², or about 8 to about 9 N/m². In still another instance, the elastomeric positioning aide has a tear-strength of about 15 to about 30 N/m², or about 20 to 25 N/m².

Preferably, the elastomeric positioning aide consists of a material that is x-ray transparent. In one example, the elastomeric positioning aide includes ELASTOSIL LR 3003/70. In another example, the elastomeric positioning aide consists essentially of ELASTOSIL LR 3003/70 (that is, the elastomeric positioning aide is composed of ELASTOSIL LR 3003/70 and those materials that do not materially affect the basic and novel characteristic(s) of the boot, including but not limited to the Shore A Hardness, the tensile strength, and the tear-strength). In still another example, the elastomeric positioning aide consists of ELASTOSIL LR 3003/70.

Furthermore, the elastomeric positioning aide preferably has a wall thickness of 0.5 to 10 mm, or about any one or more of 0.5 mm, 1 mm, 2 mm, 3 mm, 4 mm, 5 mm, 6 mm, 7 mm, 8 mm, 9 mm, and/or 10 mm. Notably portions of the elastomeric positioning aide may be greater wall thicknesses than other portions (e.g., corners and edges may be thicker then the portion that covers the exposure surface).

In another instance, the elastomeric positioning aide is autoclavable. That is, the elastomeric positioning aide retains its form and characteristics after being processed through an autoclave (e.g., exposure to steam at a temperature greater than 100° C.).

In still another instance, the veterinary dental sensor, including the digital x-ray sensor and the boot can be enwrapped in or covered by a sensor cover (e.g., a plastic bag) that gives some additional protection to the veterinary dental sensor from biological fluids.

A third embodiment is a veterinary dental process that includes using the above described veterinary dental sensor to image a veterinary patient's tooth/teeth. The process can include positioning a veterinary dental sensor comprising elastomeric positioning aide covering a portion of a digital x-ray sensor against a tooth of a veterinary patient, where the elastomeric positioning aide holds the position of the veterinary dental sensor. Then exposing the veterinary dental sensor to x-rays to image the tooth and then withdrawing the veterinary dental sensor from the veterinary patient. Typically, during veterinary dentistry, the veterinary patient is under general anesthesia. As noted above, the elastomeric positioning aide is, preferably, pigmented to a nonbiological color, where the nonbiological color clarifies the position of the veterinary dental sensor during the exposure to x-rays.

The foregoing description is given for clearness of understanding only, and no unnecessary limitations should be understood therefrom, as modifications within the scope of the invention may be apparent to those having ordinary skill in the art. While the compositions and methods of this invention have been described in terms of preferred embodiments, it will be apparent to those of skill in the art that variations may be applied to the compositions and/or methods in the steps or in the sequence of steps of the method described herein without departing from the concept, spirit and scope of the invention. More specifically, it will be apparent that certain agents that are both chemically and physically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. An elastomeric positioning aide comprising:
a silicon rubber boot that includes an interior surface, an exterior surface and an orifice connecting the exterior surface to the interior surface,
the orifice adapted to elastically expand and pass a digital x-ray sensor therethrough whereafter the digital x-ray sensor is adjacent to the interior surface and has a connection to a digital recording system passing through the orifice,
the interior surface adapted to adhere to an exposure surface of the digital x-ray sensor and to grip edges of the digital x-ray sensor,
the exterior surface adapted to grip a tooth of a veterinary patient and provide visual identification of the digital x-ray sensor within the veterinary patient's mouth.

2. The elastomeric positioning aide of claim 1, wherein the silicon rubber boot is composed of a silicon rubber having a Shore A Hardness of 60 to 90, a tensile strength of about 6 to about 10 N/m², and a tear-strength of about 15 to about 30 N/m².

3. The elastomeric positioning aide of claim 1, wherein the interior surface has interior volume dimensions that are the same as or less than 2 mm smaller than exterior surface dimensions of the digital x-ray sensor to which it is adapted to adhere to.

4. A veterinary dental sensor comprising:
a digital x-ray sensor having an exposure surface, a plurality of perimeter surfaces, and a connection surface opposite the exposure surface and adapted to communication with a digital recordation system;
an elastomeric positioning aide covering the exposure surface and the plurality of perimeter surfaces; the elastomeric positioning aide frictionally adhered to the sensor;
the elastomeric positioning aide having an internal surface adapted to receive the digital x-ray sensor and an external surface adapted to grip a tooth of a veterinary patient.

5. The veterinary dental sensor of claim 4, wherein the elastomeric positioning aide is pigmented to a nonbiological color.

6. The veterinary dental sensor of claim 5, wherein the nonbiological is blue.

7. The veterinary dental sensor of claim 4, wherein the elastomeric positioning aide comprises a silicon rubber.

8. The veterinary dental sensor of claim 7, wherein the elastomeric positioning aide has a Shore A Hardness of 60 to 90; of 60 to 80, of 65 to 75, or to about 70.

9. The veterinary dental sensor of claim 4, wherein the elastomeric positioning aide has a tensile strength of about 6 to about 10 N/m², about 7 to about 9 N/m², or about 8 to about 9 N/m².

10. The veterinary dental sensor of claim 4, wherein the elastomeric positioning aide has a tear-strength of about 15 to about 30 N/m², or about 20 to 25 N/m².

11. The veterinary dental sensor of claim 4, wherein the elastomeric positioning aide consists essentially of ELASTOSIL LR 3003/70.

12. The veterinary dental sensor of claim 11, wherein the elastomeric positioning aide consists of ELASTOSIL LR 3003/70.

13. The veterinary dental sensor of claim 4, wherein the elastomeric positioning aide having a wall thickness of 0.5 to 10 mm.

14. The veterinary dental sensor of claim 4, wherein the elastomeric positioning aide is autoclavable.

15. The veterinary dental sensor of claim 4, further comprising a sensor cover enwrapping the digital x-ray sensor and carrying the elastomeric positioning aide.

16. A veterinary dental process comprising:
positioning a veterinary dental sensor comprising elastomeric positioning aide covering a portion of a digital x-ray sensor against a tooth of a veterinary patient, where the elastomeric positioning aide holds the position of the veterinary dental sensor;
exposing the veterinary dental sensor to x-rays to image the tooth; and then
withdrawing the veterinary dental sensor from the veterinary patient.

17. The veterinary process of claim 16, wherein the veterinary patient is under general anesthesia.

18. The veterinary process of claim 16, wherein the elastomeric positioning aide is pigmented to a nonbiological color and wherein the nonbiological color clarifies the position of the veterinary dental sensor during the exposure to x-rays.
